(19) United States
(12) Patent Application Publication
(10) Pub. No.: US 2004/0192586 A1
(43) Pub. Date: Sep. 30, 2004

(54) Materials and methods relating to anti-inositolphosphoglycan antibodies

(75) Inventors: Varela-Nieto Isabel, Madrid (ES); Jose Mato, Navarra (ES); Jesus Prieto, Pamplona (ES); Phillip Williams, Oxford (GB); Thomas William Rademacher, Oxford (GB)

Correspondence Address: QUINE INTELLECTUAL PROPERTY LAW GROUP, P.C., P O BOX 458, ALAMEDA, CA, 94501, US

(73) Assignee: Rodaris Pharmaceuticals Limited

(21) Appl. No.: 10/817,247

(22) Filed: Apr. 1, 2004

Related U.S. Application Data
(63) Continuation of application No. 10/439,851, filed on May 15, 2003, now abandoned.
Application No. 10/439,851 is a continuation of application No. 09/646,468, filed on Oct. 26, 2000, now abandoned.
Application No. 09/646,468 is a 371 of international application No. PCT/GB99/00844, filed on Mar. 18, 1999.

(30) Foreign Application Priority Data
Mar. 18, 1998 (GB) 9805739.1
May 21, 1998 (GB) 9811000.0

(51) Int. Cl. A61K 38/00

(52) U.S. Cl. 514/2

(57) ABSTRACT

The present invention relates to anti-IPG antibodies, and in particular monoclonal antibodies produced by hybridoma cell lines 2F7. 2D1 and 5H6, and the use of these and other similar antibodies in the treatment and diagnosis of pre-eclampsia or diabetes, especially type I diabetes. A method of producing anti-IPG antibodies by immunising an animal with IPG unconjugated to an immunogenic carrier is also disclosed.

### FIELD OF THE INVENTION

The present invention relates to antibodies capable of specifically binding to inositolphosphoglycans (IPGs), to methods for making these antibodies and their diagnostic and therapeutic uses, in particular in assays.

### BACKGROUND OF THE INVENTION

Many of the actions of growth factors on cells are thought to be mediated by a family of inositol phosphoglycan (IPG) second messengers (Rademacher et al, 1994). It is thought that the source of IPGs is a “free” form of glycosyl phosphatidylinositol (GPI) situated in cell membranes. IPGs are thought to be released by the action of phosphatidylinositol-specific phospholipases following ligation of growth factors to receptors on the cell surface. There is evidence that IPGs mediate the action of a large number of growth factors including insulin, nerve growth factor, hepatocyte growth factor, insulin-like growth factor I (IGF-I), fibroblast growth factor, transforming growth factor β, the action of IL-2 on B-cells and T-cells, ACTH signalling of adrenocortical cells, IgE, FSH and hCG stimulation of granulosa cells, thyrotropin stimulation of thyroid cells, cell proliferation in the early developing ear and rat mammary gland.

Soluble IPG fractions have been obtained from a variety of animal tissues including rat tissues (liver, kidney, muscle brain, adipose, heart) and bovine liver. IPG biological activity has also been detected in malaria parasitized red blood cells (RBC) and mycobacteria. We have divided the family of IPG second messengers into distinct A and P-type subfamilies on the basis of their biological activities. In the rat, release of the A and P-type mediators has been shown to be tissue-specific (Kunjara et al, 1995).

There are some references in the prior art to the production of polyclonal antibodies having anti-IPG specificity (see Romero et al, 1990; Huang et al, 1993; Nestler et al, 1991; Represa et al, 1991). In these papers, a single polyclonal antibody is described which was raised against the GPI-anchor of the variant surface protein (VSG) of Trypanosoma brucei. The authors were forced to use this cross-reacting antigen for obtaining anti-IPG antibodies as soluble IPGs had not been purified and characterised, and so were not available for raising specific anti-IPG antibodies directly. The polyclonal antibodies produced using this immunisation protocol react also with the GPI-anchor moieties of GPI-anchor containing proteins. The papers report that this polyclonal antibody inhibits the action of insulin on human placental steroidogenesis, inhibits the action of insulin on muscle cells, inhibits the action of insulin on rat diaphragm and inhibits the action of nerve growth factor on chick cochleovestibular ganglia (CVG).

### SUMMARY OF THE INVENTION

The present invention relates to the production of polyclonal and monoclonal antibodies specific for P and A-type inositolphosphoglycans (IPGs), and in particular to the first production of monoclonal antibodies capable of specifically binding to these substances. The present invention also relates to the uses of anti-IPG antibodies, especially in the diagnosis of diabetes and pre-eclampsia.

In the field of diabetes, a central problem concerns the identification of patients at risk of developing type I diabetes. This condition typically arises between the ages of 5 and 11 and is caused by a patient's immune system attacking β-cells in the pancreas, destroying these cells and causing inflammation. If patients at risk of developing type I diabetes could be identified before this attack takes place, it opens up the possibility of ameliorating or preventing the attack by shutting down the pancreas during the period of risk by providing the patients with insulin or an alternative treatment for diabetes. After the period of risk is over, e.g. after puberty, this treatment could cease, and the patient's pancreas allowed to resume making insulin. Currently, there is no reliable method of identifying such patients.

As for pre-eclampsia, at present there is no simple assay for the diagnosis of this condition which affects 10-12% of all pregnancies, causing maternal endothelial dysfunction and problems with activation of the clotting system, increased vascular permeability and ischaemia in maternal organs secondary to vasoconstriction.

Accordingly, in a first aspect, the present invention provides a hybridoma cell line which produces anti-inositolphosphoglycan (IPG) monoclonal antibodies, wherein the cell line is selected from hybridoma cell lines 2F7, 2D1 and 5H6 deposited at European Collection of Cell Cultures (ECACC) under accession numbers 98051201, 98031212 and 98030901.

In a further aspect, the present invention provides anti-IPG monoclonal antibodies as obtainable from the above hybridoma cell lines or which are capable of binding to an epitope of an IPG which is bound by a monoclonal antibody produced by one or more of the deposited cell lines. In particular, the antibodies were produced following immunisation with A-type IPGs as obtainable from rat liver and yet bind to P-type IPGs as obtainable from human placenta. This surprising result indicates that the antibodies recognise a common epitope on these IPGs. Thus, in a preferred embodiment, the present invention provides antibodies capable of binding to the same or substantially the same epitope of a P or A-type IPG as one of the above monoclonal antibodies.

Preferably, the antibodies of the invention do not bind to the common reactive determinant (CRD) of GPI anchored proteins. Experiments demonstrating this are set out below in example 6.

In some embodiments, the antibodies are neutralising antibodies and can be used as antagonists to reduce or inhibit one or more of the biological properties of the P or A-type IPGs.

In further aspects, the present invention provides the above antibodies for use in a method of medical treatment and pharmaceutical compositions comprising the antibodies.

In a further aspect, the present invention provides the use of the above antibodies for the preparation of a composition for use in a diagnostic assay. In preferred embodiments, the assays are for the diagnosis of pre-eclampsia or diabetes. In a preferred embodiment, the use is for the diagnosis of patients at risk of developing type I diabetes.

In a preferred embodiment, the present invention provides a method for diagnosing patients at risk of developing type I diabetes, the method comprising:

(a) contacting a biological sample from the patient with an antibody capable of specifically binding P and/or A-type IPGs; and,

(b) determining the binding of IPGs in the sample to the anti-IPG antibody.

Optionally, the method comprises the further step of administering insulin and/or P-type IPGs and/or A-type IPGs to a patient shown in the assay to have or be at risk of developing type I diabetes, e.g. for the period of risk.

In an alternative embodiment, the present invention provides a method for diagnosing pre-eclampsia in a patient, the method comprising:

(a) contacting a biological sample from the patient with one of the above anti-IPG antibodies; and,

(b) determining the binding of IPGs in the sample to the anti-IPG antibodies.

Optionally, the method comprises the further step of administering a P-type IPG antagonist to a patient shown in the assay to have or be at risk of developing pre-eclampsia.

Exemplary assay protocols are described in more detail below. Typically, the binding of IPGs in the sample to the anti-IPG antibody is compared to the amount of binding that takes place in standards, e.g. from a non-diabetic patient, or from a person known to have a particular type of diabetes, e.g. a patient known to have developed type I diabetes or have pre-eclampsia.

In one embodiment, the assay can be carried out by providing the patient with a carbohydrate load (e.g. glucose) and measuring the IPG response following the load over time. The IPG profiles or levels determined from a patient sample can then be compared with the profiles or levels obtained from standards to make the diagnosis. Conveniently, the antibody is selected from the group of deposited monoclonal anti-IPG antibodies, or antibodies prepared using the methods described herein.

In a further aspect, the present invention provides the use of an antibody described herein in the immunopurification of P or A-type IPGs.

In a further aspect, the present invention provides a method of producing antibodies specific for P and/or A-type IPGs, the method comprising immunising an animal with one or more soluble P or A-type IPGs to elicit an antibody response, wherein the IPGs are not conjugated to an immunogenic carrier.

The inventors have surprisingly found that despite the fact that IPGs are small molecules and carbohydrates, it is possible to raise antibodies by immunising animals with them. Further, the fact that conjugation of the IPGs to a carrier is not required means that epitopes on the IPGs are not blocked or altered by the conjugation reaction, allowing antibodies to be raised to a full complement of epitopes on the IPGs. The method is applicable to the production of both polyclonal and monoclonal antibodies, as is demonstrated by the examples below. Preferably, the animal is immunised via an intraperitoneal route using a soluble form of the IPG.

Embodiments of the present invention will now be described by way of example and not by limitation with reference to the accompanying drawings.

### BRIEF DESCRIPTION OF THE DRAWINGS

FIG. 1 shows a dose-response curve of absorbance plotted against IPG concentration using anti-IPG antibodies in a Sandwich ELISA test.

FIG. 2 shows how IPG levels in human serum vary with time after an oral load of glucose (75 g) in a control patient without a familiar history of type I diabetes.

FIG. 3 shows how IPG levels in human serum vary with time after an oral load of glucose (75 g) in a patient with a familiar history of type I diabetes

FIG. 4 shows how IPG levels in human serum vary with time after an oral load of glucose (75 g) in a patient with carbohydrate intolerance.

FIGS. 5 and 6 show investigations into the properties of monoclonal antibody 2D1 in chick cochleovestibular ganglia (CVG) culture.

FIG. 7 shows the results of experiments comparing the binding specificity of monoclonal antibody 5H6, a polyclonal antibody raised against the IPGs and a polyclonal antibody raised against the cross reactive determinant (CRD) common to GPI anchored proteins.

FIG. 8 shows the results of a PDH phosphatase assay demonstrating that monoclonal antibody 5H6 is capable of inhibiting the action of P-type IPG from rat liver.

FIG. 9 shows the results of a pre-eclampsia ELISA assay employing anti-IPG antibodies of the invention to measure urine IPG levels.

FIG. 10 shows the results of a blinded pre-eclampsia assay using anti-IPG monoclonal antibody 2D1 to measure urine IPG levels.

FIG. 11 shows the relationship between the IPG assay in pre-eclamptic urine using anti-IPG monoclonal antibody 2D1 and platelet counts.

FIG. 12 shows the relationship between the IPG assay in pre-eclamptic urine using anti-IPG monoclonal antibody 2D1 and plasma AST activity.

### DETAILED DESCRIPTION

IPGs and IPG Analogues

Studies have shown that A-type mediators modulate the activity of a number of insulin-dependent enzymes such as cAMP dependent protein kinase (inhibits), adenylate cyclase (inhibits) and cAMP phospho-diesterases (stimulates). In contrast, P-type mediators modulate the activity of insulin-dependent enzymes such as pyruvate dehydrogenase phosphatase (stimulates), glycogen synthase phosphatase (stimulates) and cAMP dependent protein kinase (inhibits). The A-type mediators mimic the lipogenic activity of insulin on adipocytes, whereas the P-type mediators mimic the glycogenic activity of insulin on muscle. Both A and P-type mediators are mitogenic when added to fibroblasts in serum free media. The ability of the mediators to stimulate fibroblast proliferation is enhanced if the cells are transfected with the EGF-receptor. A-type mediators can stimulate cell proliferation in the CVG.

Soluble IPG fractions having A-type and P-type activity have been obtained from a variety of animal tissues including rat tissues (liver, kidney, muscle brain, adipose, heart) and bovine liver. A and P-type IPG biological activity has also been detected in human liver and placenta, malaria parasitized RBC and mycobacteria. The ability of a polyclonal cross-reacting anti-inositolglycan antibody to inhibit insulin action on human placental cytotrophoblasts and BC3H1 myocytes or bovine-derived IPG action on rat diaphragm and chick cochleovestibular ganglia suggests cross-species conservation of many structural features. However, it is important to note that although the prior art includes these reports of A and P-type IPG activity in some biological fractions, the purification or characterisation of the agents responsible for the activity was not disclosed until it was reported in the references below.

A-type substances are cyclitol-containing carbohydrates, also containing Zn²⁺ ion and optionally phosphate and having the properties of regulating lipogenic activity and inhibiting cAMP dependent protein kinase. They may also inhibit adenylate cyclase, be mitogenic when added to EGF-transfected fibroblasts in serum free medium, and stimulate lipogenesis in adipocytes.

P-type substances are cyclitol-containing carbohydrates, also containing Mn²⁺ and/or Zn²⁺ ions and optionally phosphate and having the properties of regulating glycogen metabolism and activating pyruvate dehydrogenase phosphatase. They may also stimulate the activity of glycogen synthase phosphatase, be mitogenic when added to fibroblasts in serum free medium, and stimulate pyruvate dehydrogenase phosphatase.

Methods for obtaining A-type and P-type IPGs are set out in detail in Caro et al, 1997, and in WO98/11116 and WO98/11117. Methods for obtaining the free GPI precursors of the A and P-type IPGs are set out below.

Glycolipids were partially purified from rat liver membranes following the method described in Mato et al (1987), with minor modifications (Varela-Nieto et al, 1993). The membrane fraction was obtained by sequential centrifugation from 300 g of material. Total lipids were obtained by chloroform/methanol extraction followed by the removal of nonpolar lipids. GPI was separated from other phospholipids by sequential acid/base silica gel G60 t.l.c. (see below). Lipids were extracted from the t.l.c. plate with methanol, dried, applied to a Sep-Pak C18 cartridge, eluted with methanol, and dried. GPI was spotted onto the origin of a silica gel G60 t.l.c. plate which was developed twice in an acidic solvent system [chloroform:acetone:methanol:acetic acid:water (50:20:10:10:5, by volume)] and in a basic solvent system [chloroform:methanol:ammonium hydroxide:water (45:45:3:5:10, by volume)]. Alternatively, GPI molecules were further resolved by double-dimension t.l.c. as described in Clemente et al (1995) and Avila et al (1992) or by using high-performance (HP)-t.l.c. plates developed in chloroform:methanol:ammonium hydroxide:water (40:45:3:5:15, by volume) as described in Gaulton (1991). Lipid migration was calibrated in parallel with phospholipid standards that were detected by staining with iodine. In order to generate free IPGs from the purified GPI, the GPI was incubated with 1 unit of PI-PLC from Bacillus thuringiensis for 3 hours according to the manufacturer's instructions. At the end of the incubation, the reaction was terminated by loading the sample onto a Sep-Pak C18 cartridge. The water eluate (5 ml) was dried.

Antibodies

Antibodies capable of specifically binding to P and A-type IPGs are disclosed herein. These antibodies can be modified using techniques which are standard in the art. Antibodies similar to those exemplified for the first time here can also be produced using the teaching herein in conjunction with known methods. These methods of producing antibodies include immunising a mammal (e.g. mouse, rat, rabbit, horse, goat, sheep or monkey) with the IPG or a fragment thereof. Antibodies may be obtained from immunised animals using any of a variety of techniques known in the art, and screened, preferably using binding of antibody to antigen of interest. Isolation of antibodies and/or antibody-producing cells from an animal may be accompanied by a step of sacrificing the animal.

As an alternative or supplement to immunising a mammal with an IPG, an antibody specific for an IPG may be obtained from a recombinantly produced library of expressed immunoglobulin variable domains, e.g. using lambda bacteriophage or filamentous bacteriophage which display functional immunoglobulin binding domains on their surfaces; for instance see WO92/01047. The library may be naive, that is constructed from sequences obtained from an organism which has not been immunised with any of the IPGs (or fragments), or may be one constructed using sequences obtained from an organism which has been exposed to the antigen of interest.

The term “monoclonal antibody” as used herein refers to an antibody obtained from a substantially homogenous population of antibodies, i.e. the individual antibodies comprising the population are identical apart from possible naturally occurring mutations that may be present in minor amounts. Monoclonal antibodies can be produced by the method first described by Kohler and Milstein, Nature, 256:495, 1975 or may be made by recombinant methods, see Cabilly et al, U.S. Pat. No. 4,816,567, or Mage and Lamoyi in Monoclonal Antibody Production Techniques and Applications, pages 79-97, Marcel Dekker Inc, New York, 1987.

In the hybridoma method, a mouse or other appropriate host animal is immunised with the antigen by subcutaneous, intraperitoneal, or intramuscular routes to elicit lymphocytes that produce or are capable of producing antibodies that will specifically bind to the IPG used for immunisation. Alternatively, lymphocytes may be immunised in vitro. Lymphocytes then are fused with myeloma cells using a suitable fusing agent, such as polyethylene glycol, to form a hybridoma cell, see Goding, Monoclonal Antibodies: Principles and Practice, pp. 59-103 (Academic Press, 1986). Immunisation with soluble IPG and via the intraperitoneal route shown in the examples was surprisingly effective in producing antibodies specific for IPGs.

The hybridoma cells thus prepared can be seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high level expression of antibody by the selected antibody producing cells, and are sensitive to a medium such as HAT medium.

Culture medium in which hybridoma cells are growing is assayed for production of monoclonal antibodies directed against the IPGs. Preferably, the binding specificity is determined by enzyme-linked immunoabsorbance assay (ELISA). The monoclonal antibodies of the invention are those that specifically bind to either or both P and A-type IPGs.

The epitope bound by the antibodies can be mapped using synthetic compounds known to bind to one of the deposited antibodies, e.g. to see whether an antibody has substantially the same or the same binding specificity as the deposited monoclonal antibodies 2F7, 2D1 or 5H6. This can also be carried out in competitive studies to determine whether a given anti-IPG antibody competes with one of the deposited antibodies for a particular IPG epitope. Thus, the present invention includes antibodies which are capable of binding to an IPG epitope bound by an exemplified antibody. Preferably, this epitope is present on both rat liver A-type IPG and human placenta P-type IPG. The exemplified monoclonal raised using rat liver A-type IPGs are surprisingly effective in assays for the diagnosis of pre-eclampsia, as shown in the examples below.

In a preferred embodiment of the invention, the monoclonal antibody will have an affinity which is greater than micromolar or greater affinity (i.e. an affinity greater than 10⁻⁶ mol) as determined, for example, by Scatchard analysis, see Munson & Pollard, Anal. Biochem., 107:220, 1980.

After hybridoma cells are identified that produce neutralising antibodies of the desired specificity and affinity, the clones can be subcloned by limiting dilution procedures and grown by standard methods. Suitable culture media for this purpose include Dulbecco's Modified Eagle's Medium or RPM1-1640 medium. In addition, the hybridoma cells may be grown in vivo as ascites tumours in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Nucleic acid encoding the monoclonal antibodies of the invention is readily isolated and sequenced using procedures well known in the art, e.g. by using oligonucleotide probes that are capable of binding specifically to genes encoding the heavy and light chains of murine antibodies. The hybridoma cells of the invention are a preferred source of nucleic acid encoding the antibodies or fragments thereof. Once isolated, the nucleic acid is ligated into expression or cloning vectors, which are then transfected into host cells, which can be cultured so that the monoclonal antibodies are produced in the recombinant host cell culture.

Hybridomas capable of producing antibody with desired binding characteristics are within the scope of the present invention, as are host cells containing nucleic acid encoding antibodies (including antibody fragments) and capable of their expression. The invention also provides methods of production of the antibodies including growing a cell capable of producing the antibody under conditions in which the antibody is produced, and preferably secreted.

Antibodies according to the present invention may be modified in a number of ways. Indeed the term “antibody” should be construed as covering any binding substance having a binding domain with the required specificity. Thus, the invention covers antibody fragments, derivatives, functional equivalents and homologues of antibodies, including synthetic molecules and molecules whose shape mimics that of an antibody enabling it to bind an antigen or epitope, here a P or A-type inositolphosphoglycan.

Examples of antibody fragments, capable of binding an antigen or other binding partner, are the Fab fragment consisting of the VL, VH, Cl and CH1 domains; the Fd fragment consisting of the VH and CH1 domains; the Fv fragment consisting of the VL and VH domains of a single arm of an antibody; the dAb fragment which consists of a VH domain; isolated CDR regions and F(ab′)₂ fragments, a bivalent fragment including two Fab fragments linked by a disulphide bridge at the hinge region. Single chain Fv fragments are also included.

A hybridoma producing a monoclonal antibody according to the present invention may be subject to genetic mutation or other changes. It will further be understood by those skilled in the art that a monoclonal antibody can be subjected to the techniques of recombinant DNA technology to produce other antibodies, humanised antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP-A-0184187, GB-A-2188638 or EP-A-0239400. Cloning and expression of chimeric antibodies are described in EP-A-0120694 and EP-A-0125023.

Pharmaceutical Compositions

The antibodies of the invention can be formulated in pharmaceutical compositions. An example of this is employing antibodies specific for P-type IPG in the treatment of pre-eclampsia, a condition associated with elevated levels of P-type IPGs. Protocols for the treatment of pre-eclampsia using P-type IPG antagonists are described in copending WO98/10791.

The pharmaceutical compositions may comprise, in addition to one or more of the antibodies, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, or intraperitoneal routes.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as sodium chloride injection, Ringer's injection, lactated Ringer's injection. Preservatives, stabilisers, buffers, antioxidants and/or other additives may be included, as required.

Preferably, the pharmaceutically useful compound according to the present invention is given to an individual in a “prophylactically effective amount” or a “therapeutically effective amount” (as the case may be, although prophylaxis may be considered therapy), this being sufficient to show benefit to the individual. Typically, this will be to cause a therapeutically useful effect in the patient, e.g. using the antibodies to antagonise one or more of the biological activities of P and/or A-type IPGs to a beneficial extent. The actual amount of the antibodies administered, and rate and time-course of administration, will depend on the nature and severity of the condition being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Oslo, A. (ed), 1980.

By way of example, depending on the type and severity of the IPG related condition, the composition can be administered to provide an initial dose of about 0.01 to 20 mg, more preferably 0.02 to 10 mg, of antibody/kg of patient weight. As mentioned above, other dosing regimens and the determination of appropriate amount of the antibodies for inclusion in the compositions can be readily determined by those skilled in the art.

Immunoassays

The antibodies described above can be employed in the diagnostic aspects of the invention in a wide variety of different assay formats. The antibodies can be used as binding agents capable of specifically binding to IPGs or as developing agents for determining the fraction of binding sites of a binding agent occupied by analyte after exposure to a test sample.

In some instances, employing the antibodies, particularly as developing agents in assays, involves tagging them with a label or reporter molecule which can directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding antibody and reporter molecule. Any method known in the art for separately conjugating the antibody to the detectable moiety may be employed, including those methods described by Hunter et al, Nature 144:945, 1962; David et al, Biochemistry 13:1014, 1974; Pain et al, J. Immunol. Meth. 40:219, 1981; and Nygren, J Histochem. and Cytochem. 30:407, 1982.

One favoured mode is by covalent linkage of each antibody with an individual fluorochrome, phosphor or laser dye with spectrally isolated absorption or emission characteristics. Suitable fluorochromes include fluorescein, rhodamine, luciferin, phycoerythrin and Texas Red. Suitable chromogenic dyes include diaminobenzidine. Other detectable labels include radioactive isotopic labels, such as ³H, ¹⁴C, ³²P, ³⁵S, ¹²⁶I, or ^{99m}Tc, and enzyme labels such as alkaline phosphatase, β-galactosidase or horseradish peroxidase, which catalyze reactions leading to detectable reaction products and can provide amplification of signal.

Other reporters include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules may be enzymes which catalyze reactions that develop or change colour or cause changes in electrical properties, for example. They may be molecularly excitable, such that electronic transitions between energy states result in characteristic spectral absorptions or emissions. They may include chemical entities used in conjunction with biosensors.

Alternatively or additionally, the antibodies can be employed as binding agents, relying on the fact that the antibodies are capable of specifically binding the IPGs in preference to other substances present in the sample. Conveniently, the binding agent(s) are immobilised on solid support, e.g. at defined locations, to make them easy to manipulate during the assay. This can be achieved using techniques well known in the art such as physisorption or chemisorption, e.g. employing biotin/avidin or biotin/streptavidin to chemically link the antibodies to the solid support. Generally, the sample is contacted with the binding agent(s) under appropriate conditions so that P and A-type IPGs present in the sample can bind to the binding agent(s). The fractional occupancy of the binding sites of the binding agent(s) can then be determined using a developing agent or agents.

As mentioned above, the developing agents are labelled (e.g. with radioactive, fluorescent or enzyme labels) so that they can be detected using techniques well known in the art. Thus, radioactive labels can be detected using a scintillation counter or other radiation counting device, fluorescent labels using a laser and confocal microscope, and enzyme labels by the action of an enzyme label on a substrate, typically to produce a colour change. The developing agent(s) can be used in a competitive method in which the developing agent competes with the analyte for occupied binding sites of the binding agent, or non-competitive method, in which the labelled developing agent binds analyte bound by the binding agent or to occupied binding sites. Both methods provide an indication of the fraction of the binding sites occupied by the analyte, and hence the concentration of the analyte in the sample, e.g. by comparison with standards obtained using samples containing known concentrations of the analyte.

Diagnostic assays can be carried out with a biological sample from a patient. These samples can be used directly or in some instances may require treatment prior to carrying out the assay, e.g. to remove potentially interfering substances in the sample. Examples of suitable biological samples are blood, urine, sweat, tissue or serum.

In one embodiment, the present invention concerns a method a diagnosing patients at risk of developing type I diabetes, the method comprises the steps of:

(a) contacting a biological sample obtained from the patient with a solid support having immobilised thereon antibodies capable of specifically binding to P and/or A-type IPGs;

(b) contacting the solid support with a labelled developing agent capable of binding to unoccupied binding sites of the antibodies, bound IPGs or occupied binding sites of the antibodies; and,

(c) detecting the label of the developing agent specifically binding in step (b) to obtain a value representative of the concentration of the IPGs in the sample.

In a further embodiment, the present invention provides a method for diagnosing pre-eclampsia in a patient, the method comprising:

(a) contacting a biological sample from the patient with an anti-IPG antibody of any one of claims 2 to 5; and,

(b) determining the binding of IPGs in the sample to the anti-IPG antibody.

Typically, the binding of IPGs in the sample to the anti-IPG antibody is compared to the amount of binding that takes place in controls, e.g. from a non-diabetic, or from a person known to have a particular type of diabetes, e.g. a patient known to have developed type I diabetes or pre-eclamptic standards. In the protocols exemplified below, this is assessed by initially providing the patient with a glucose load and measuring the IPG response following the glucose load over time. The IPG profiles or levels determined from a sample can then be compared with the profiles or levels obtained from standards to make the diagnosis.

Thus, preferably, the method includes the further step of correlating the value representative of the concentration of the IPGs in the sample to values obtained from known standards to determine whether the patient is at risk from developing type I diabetes or has pre-eclampsia.

The antibodies of the present invention may be employed in any known assay method, such as competitive binding assays, direct and indirect sandwich assays, and immunoprecipitation assays, see Zola, Monoclonal Antibodies: A Manual of Techniques, pp 147-158 (CRC Press, Inc, 1987).

Sandwich assays involve the use of two antibodies, each capable of binding to a different immunogenic portion, or epitope, of the IPG to be detected. In a sandwich assay, the test sample analyte is bound by a first antibody which is immobilised on a solid support, and thereafter a second antibody binds to the analyte, thus forming an insoluble three part complex. The second antibody may itself be labelled with a detectable moiety (direct sandwich assays) or may be measured using an anti-immunoglobulin antibody that is labelled with a detectable moiety (indirect sandwich assay). For example, one type of sandwich assay is an ELISA assay, in which case the detectable moiety is an enzyme.

The antibodies of the invention also are useful for in vivo imaging, wherein an antibody labelled with a detectable moiety for example radioisotope and is administered to a host, preferably into the bloodstream, and the presence and location of the labelled antibody in the host is assayed. This antibody may be labelled with any moiety that is detectable in a host, whether by nuclear magnetic resonance, radiology, or other detection means known in the art.

Affinity Purification

The antibodies of the invention also are useful as affinity purification agents. In this process, the antibodies are immobilised on a suitable support, such as Sephadex® resin or filter paper, using methods well known in the art. The immobilized antibody then is contacted with a sample containing the IPGs to be purified, and thereafter the support is washed with a suitable solvent that will remove substantially all the material in the sample except the IPGs, which is bound to the immobilised antibody. Finally, the support is washed with another suitable solvent, such as glycine buffer, pH 3-5, that will release the IPGs from the antibody. This method can also be used to separate a given IPG family member from a mixture of IPGs by using an antibody capable of specifically binding that IPG in preference to other family members.

### EXAMPLE 1

Production of Polyclonal and Monoclonal Antibodies Against Inositolphosphoglycans (IPGs)

Inositolphosphoglycan (soluble form) obtained by PI-PLC treatment of GPI purified from rat liver by sequential Thin Layer Chromatography (TLC) was used to immunize New Zealand rabbits and Balb/c mice as described below. Alternatively, human IPGs could be obtained using the methods described in Caro et al, 1997.

Rabbit Immunisation Procedure

Two New Zealand rabbits were anaesthetized and then immunised with 750 μg of IPG (soluble form) mixed in 1 ml of PBS with 1 ml of complete Freund's adjuvant (CFA). The antigen-adjuvant emulsion was administered 1.5 ml by intradermal (id) injection and 0.5 ml by intramuscular (im) injection.

After one month, this protocol was repeated except that incomplete Freund's adjuvant (IFA) was used, and 1.5 ml by administered by subcutaneous (sc) injection and 0.5 ml by intramuscular (im) injection. This was repeated again on days 60, 90, 120 and 150.

Mouse Immunisation Procedure

Four female Balb/c 6 weeks old mice were immunised with 60 μg of IPG (soluble form) in 250 μl of PBS with 250 μl of CFA. The antigen-adjuvant emulsion was injected by intraperitoneal (ip) injection.

After 21 days, the injection was repeated except that IFA was used. On days 42 and 63 all the animals were injected ip with IFA. On day 84, the best responder was injected 100 μl PBS containing 60 μg of IPG intravenous (iv) and 100 μl PBS containing 60 μg of IPG (ip). After 87 days, splenocytes from best responder were fused to myleoma cells using conventional techniques. Monitor test bleeds were realized regularly.

### EXAMPLE 2

Indirect ELISA Assay

The following indirect ELISA protocol was used to screen the monoclonal antibodies.

Add 100 μl/well in all the steps.

(1) Add IPG diluted 1:800 in PBS in a F96 Polysorp Nunc-Immuno plate. Incubate at least 7 days at 4° C.

(2) Wash with PBS three times.

(3) Add a blocking reagent for ELISA (Boehringer Mannheim) in distilled water (1:10 v/v) for 2 hours at room temperature.

(4) Wash with PBS-Tween 20 (0.1%) three times.

(5) Add a purified monoclonal and/or a polyclonal antibody (diluted between 1:12.5 and 1:3200 in PBS), overnight at 4° C.

(6) Wash with PBS-Tween 20 (0.1%) three times.

(7) Add an anti-mouse IgM, biotinylated whole antibody (from goat) (Amersham) in case of the previous addition of a monoclonal antibody, or add an anti-rabbit Ig, biotinylated species-specific whole antibody (from donkey) (Amersham), in case of previous addition of a polyclonal antibody, both of them diluted 1:1000 in PBS, 1 h 30 min at room temperature.

(8) Wash with PBS-Tween 20 (0.1%) three times.

(9) Add a streptavidin-biotinylated horseradish peroxidase complex (Amersham) diluted 1:500 in PBS, 1 h 30 min at room temperature.

(10) Wash with PBS three times.

(11) Add 2,2-Azino-di-(3-ethylbenzthiazoline sulfonate (6)) diammonium salt crystals (ABTS) to buffer for ABTS (Boehringer Mannheim): Buffer for ABTS is added to distilled water (1:10 v/v). 1 mg of ABTS is added to 1 ml of diluted buffer for ABTS.

(12) Read the absorbance in a Multiskan Plus P 2.01 using a 405 nm filter in 5-15 min.

Comments:

Solvents used are phosphate-buffered saline (PBS) pH 7.2 or PBS-Tween 20 (0.1%).

Step 1: IPG (soluble form) is used in an indirect ELISA (initial concentration approximately 60 μM). Working dilutions are normally between 1:400 and 1:800.

Several incubation times have been examined. The best and more reproducible results have been obtained when incubation time is at least seven days at 4° C., probably because higher incubation times increase the number of molecules of IPG that are bound to the solid phase.

Step 3: Blocking reagent for ELISA is a registered product of Boehringer Mannheim. In order to prepare the solution we must dissolve the content (27 g) in 100 ml redist. Water at room temperature while stirring for approx 30 minutes. The concentrate is stable when stored in aliquots at −20° C. For use, dilute one aliquot in a ratio of 1:10 with redistilled water to yield a working solution containing 1% protein mixture (w/v) that was obtained by proteolytic degradation of purified gelatin, in 50 mM Tris-HCl, 150 mM NaCl, pH ca.7.4.

Step 5: A purified monoclonal and/or polyclonal antibody are used in an indirect ELISA (initial concentration approx 2 mg/ml). Working dilutions are normally between 1:100 and 1:200, getting final concentrations of 20 μg/ml and 10 μg/ml respectively).

Incubation time for rapid assays can be two hours at room temperature.

Step 7: The anti-rabbit and the anti-mouse antibodies are used at 1:1000 dilution straight from the stock bottle. The stock bottle is stored at 2-8° C. Under these conditions the product is stable for at least six months, after which any remaining solution is discarded.

1 ml of biotinylated reagent is supplied in 0.1 M phosphate-buffered saline of pH 7.5 containing 1% (w/v) bovine serum albumin and 0.05% (w/v) sodium azide.

Step 9: Streptavidin-biotinylated/HRP is used at 1:500 dilution straight from the stock bottle. The stock bottle is stored at 2-8° C. Under these conditions the product is stable for at least three months, after which any remaining solution is discarded.

The complex is supplied in 2 ml of phosphate-buffered saline pH 7.5 containing 1% (w/v) bovine serum albumin and an antimicrobial agent.

Step 11: ABTS buffer is a registered product of Boehringer Mannheim. The solution consists of sodium perborate, citric acid and disodium hydrogen phosphate. The solution is stored in aliquots of 1 ml at −20° C. For use, dilute one aliquot in a ratio of 1:10 with redistilled water.

### EXAMPLE 3

Sandwich ELISA Assay

The following ELISA protocol is provided as an example of an assay for IPGs employing the deposited monoclonal antibodies.

Add 100 μl/well in all the steps.

(1) Anti-IPG monoclonal antibody was diluted 1:100 in PBS in added to a F96 Maxisorp Nunc-Immuno plate. The plate was incubated for at least 2 days at 4° C.

(2) The plate was washed with PBS three times.

(3) Blocking reagent for Elisa (Boehringer Mannheim) in distilled water (1.5:10 v/v) was added for 2 hours at room temperature.

(4) The plate was washed with PBS-Tween 20 (0.1%) three times.

(5) Human serum diluted 1:4 in PBS was added to the plate overnight at 4° C.

(6) The plate was wash with PBS-Tween 20 (0.1%) three times.

(7) A purified polyclonal antibody (diluted 1:100 in PBS) was added overnight at 4° C.

(8) The plate was washed with PBS-Tween 20 (0.1%) three times.

(9) An anti-rabbit Ig, biotinylated species-specific whole antibody (from donkey ) (Amersham) was diluted 1:1000 in PBS, and added to the plate for 1 h 30 min at room temperature.

(10) Wash with PBS-Tween 20 (0.1%) three times.

(11) Add a streptavidin-biotinylated horseradish peroxidase complex (Amersham) diluted 1:500 in PBS, 1 h 30 mins at room temperature.

(12) The plate was washed with PBS three times.

(13) 2,2′-Azino-di-(3-ethylbenzthiazoline sulfonate (6)) diammonium salt crystals (ABTS) was made up in buffer for ABTS (Boehringer Mannheim). The buffer for ABTS was added to distilled water (1:10 v/v). 1 mg of ABTS was added to 1 ml of diluted buffer for ABTS.

(14) The absorbance was read in a Multiskan Plus P 2.01 using a 405 nm filter in 15-30 mins.

Comments:

The solvents used were phosphate-buffered saline (PBS) pH 7.2 or PBS-Tween 20 (0.1%).

In step (1): The examples employed purified monoclonal antibody 2D1 in the Sandwich ELISA assays (initial concentration 2.5 mg/ml). Working dilutions varied normally between 1:100 and 1:200, with final concentrations of 25 μg/ml and 12.5 μg/ml, respectively. The incubation time used in the assay can be varied. For rapid assays, incubation time can vary between two hours at room temperature or overnight at 4° C. However, improved results are obtained when incubation time is at least two days at 4° C. This improvement may be due to the higher incubation times increasing the number of molecules of monoclonal antibody that are bound to the solid phase.

Step (3): Blocking reagent for ELISA is a registered product of Boehringer Mannheim. In order to prepare the solution, the content (27 g) was dissolved in 100 ml redistilled water at room temperature while stirring for approximately 30 mins. The concentrate is stable when stored in aliquots at −20° C. For use, aliquots were diluted in a ratio of 1:10 with redistilled water to yield a working solution containing 1% protein mixture (w/v) that was obtained by proteolytic degradation of purified gelatin, in 50 mM Tris-HCl, 150 mm NaCl, pH ca. 7.4.

Step (5): Human serum was probed in several dilutions (1:1, 1:2, 1:4 . . . ). Working dilutions are normally between 1:2 and 1:4. IPG in several dilutions was used as a standard (positive control). Wells without serum are used as negative control.

The incubation time for rapid assays was two hours at room temperature.

Step (7): A purified polyclonal antibody was used in Sandwich ELISA (initial concentration 2 mg/ml). Working dilutions were between 1:100 and 1:200, with final concentrations of 20 μg/ml and 10 μg/ml respectively.

Step (9): The anti-rabbit was used at 1:1000 dilution straight from the stock bottle, stored at 28° C. Under these conditions the product is stable for at least six months.

1 ml of biotinylated reagent is supplied in 0.1 M phosphate-buffered saline pH 7.5 containing 1% (w/v) bovine serum albumin and 0.05% (w/v) sodium azide.

Step (11): Streptavidin-biotinylated/HRP was used at 1:500 dilution straight from the stock bottle, stored at 2-8° C. Under these conditions the product is stable for at least three months.

The complex is supplied in 2 ml of phosphate-buffered saline pH 7.5 containing 1% (w/v) bovine serum albumin and an antimicrobial agent.

Step (13): ABTS buffer is a registered product of Boehringer Mannheim. The solution consists of sodium perborate, citric acid and disodium hydrogen phosphate. The solution is stored in aliquots of 1 ml at −20° C. For use, one aliquot is diluted in a ratio of 1:10 with redistilled water.

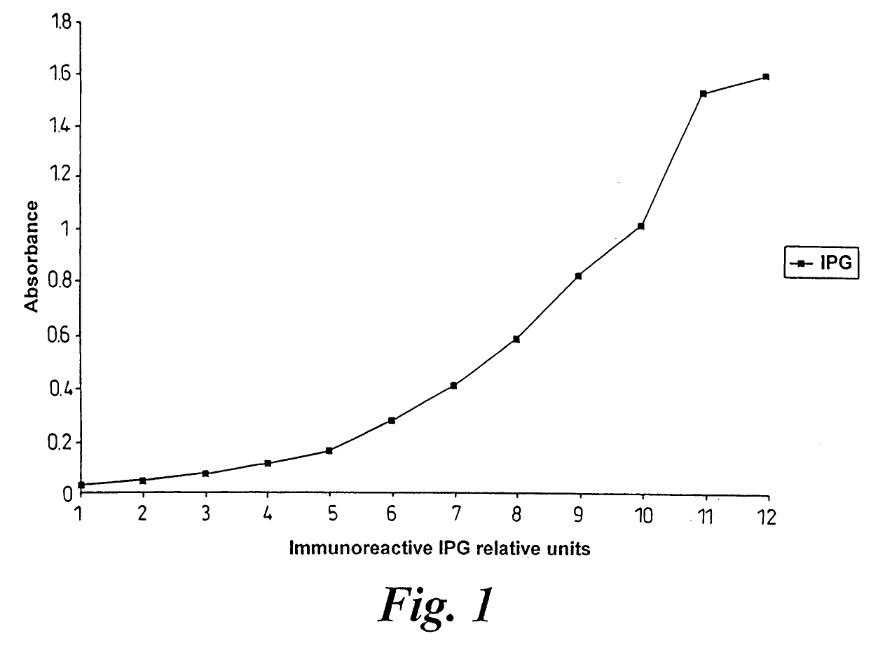

FIG. 1 shows a dose-response curve of assay response (absorbance) plotted against IPG concentration, demonstrating that the antibodies can be used to assay for IPGs.

### EXAMPLE 4

Assay for Type I Diabetes and Controls

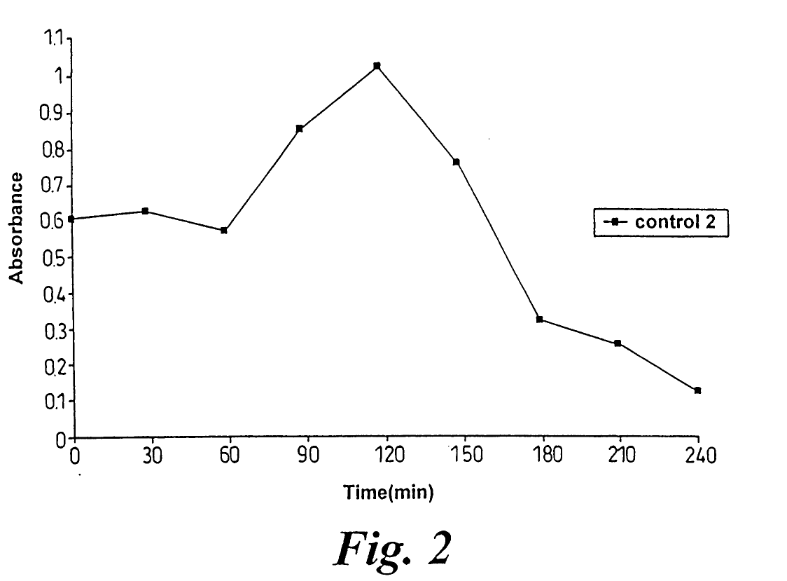

The protocol set out in example 3 was used to measure IPG levels in a different patients in response to a glucose load (75 g) FIGS. 2 to 4 plot the changes in IPG levels in the patients over time after the glucose load. The profiles shown in the figures and/or the levels of the IPGs can be used in the diagnosis of the risk of developing type I diabetes by comparing them with the profile or level obtained from a sample from a patient suspected of being at risk of developing type I diabetes. In particular, examining the change in IPG levels has the advantage that it isolates the change in IPG level caused by the glucose load from the effect of IPGs acting as second messengers for other growth factors in the patient.

FIG. 2 shows the response obtained in a control patient with no familial history of diabetes. Thus, after administration of the glucose, the IPG level in the patient rose to a maximum around 2 hours after the load, and then fell again to the background level.

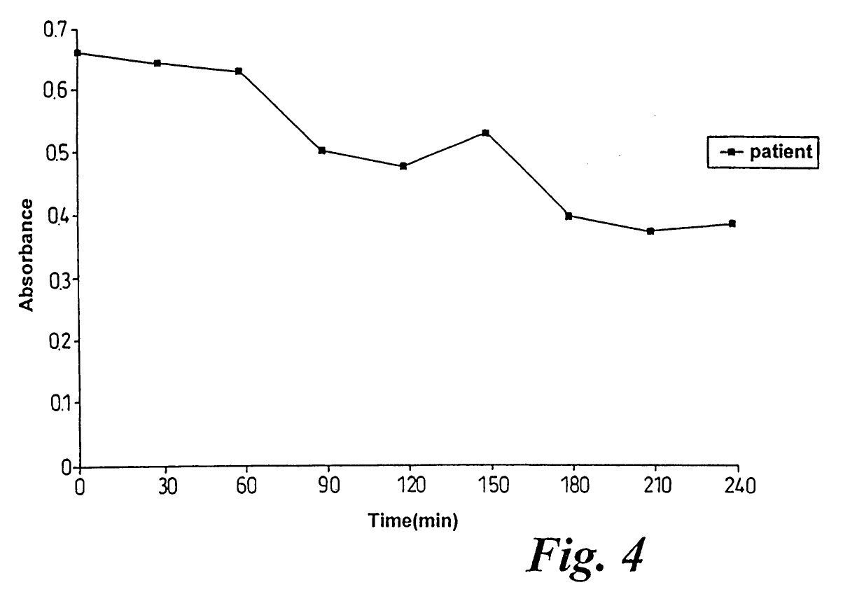

FIG. 4 shows a second control profile obtained from a patient with carbohydrate intolerance, i.e. who was unable to metabolise the glucose via insulin and subsequent IPG production. Thus, the IPG profile following the glucose load is approximately flat.

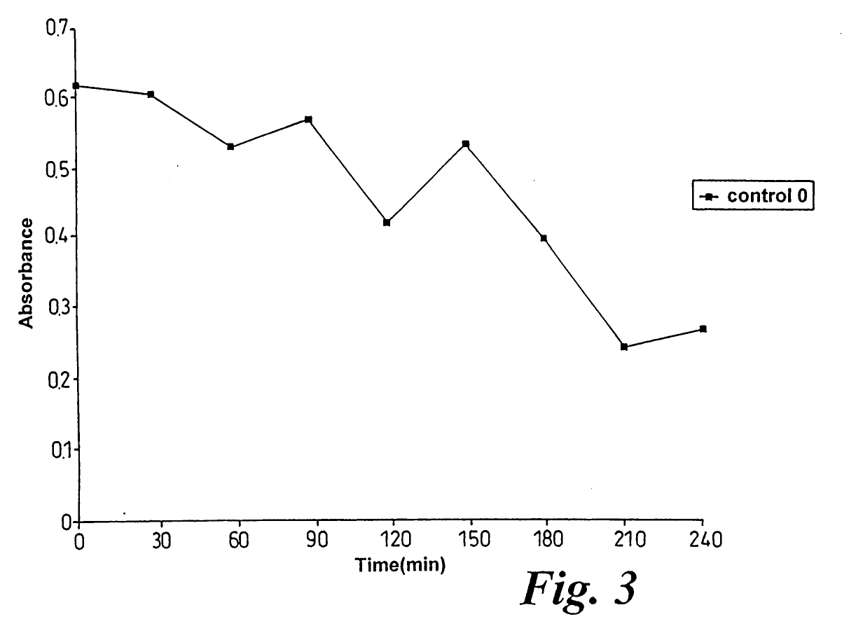

FIG. 3 shows a profile obtained from a patient with a familial history of type I diabetes. No elevation in IPG is observed indicating that there has been an inadequate release of insulin in response to the glucose challenge.

### EXAMPLE 5

Properties of the Anti-IPG Antibody 2D1

The properties of monoclonal antibody 2D1 were further investigated in CVG culture, measuring cellular proliferation in presence of 2D1 anti-IPG antibody, with and without IGF-I.

CVGs were isolated from E3.5 chicken embryos as described in Varela-Nieto et al, 1991, and cultured for 24 hours in 4-well multidishes (NUNC) in 0.25 ml of M199 culture medium with Earle's salts, 2 mM L-glutamine, 25 mM HEPES and antibiotics. Incubations were carried out at 37° C. in a humidified atmosphere with 5% CO₂.

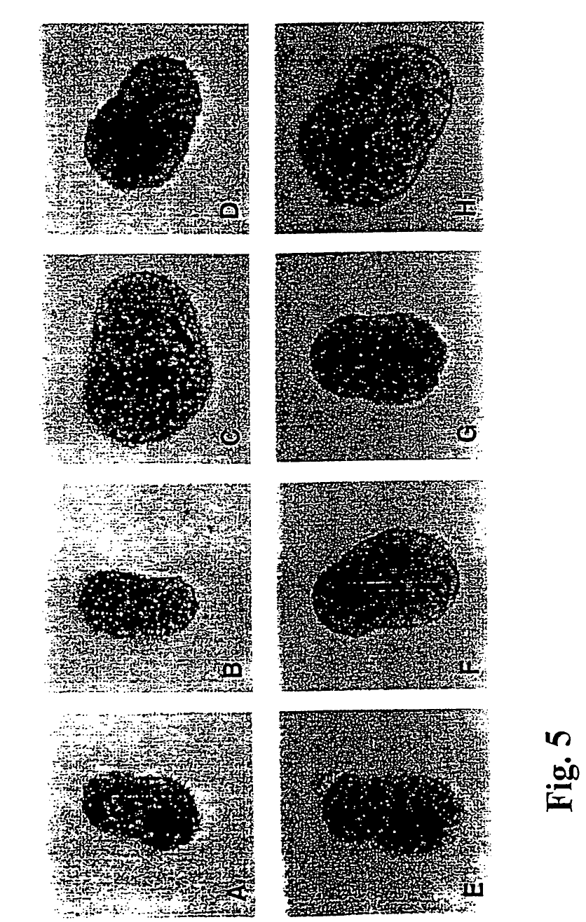

The photomicrographs of FIG. 5 show CVG proliferation under the following conditions: control medium with no additions (A), 25 μg/ml 2D1 antibody (B), 1 nm IGF-I alone (C), 1 nM IGF-1 plus 25 μg/ml 2D1 (D). The exogenous addition of 100 μM of synthetic compound β-methyl-3,4-bis(disodium phosphate) galactose (GP2) that alone do not stimulate CVG growth (E), was able to rescue the 2D1 inhibitory effect on CVG growth induced by IGF-I (F). IPG type-A (1/100), that alone has a slight effect on CVG-growth (G), completely recovered the growth inhibitory effect caused by the 2D1 antibody (H).

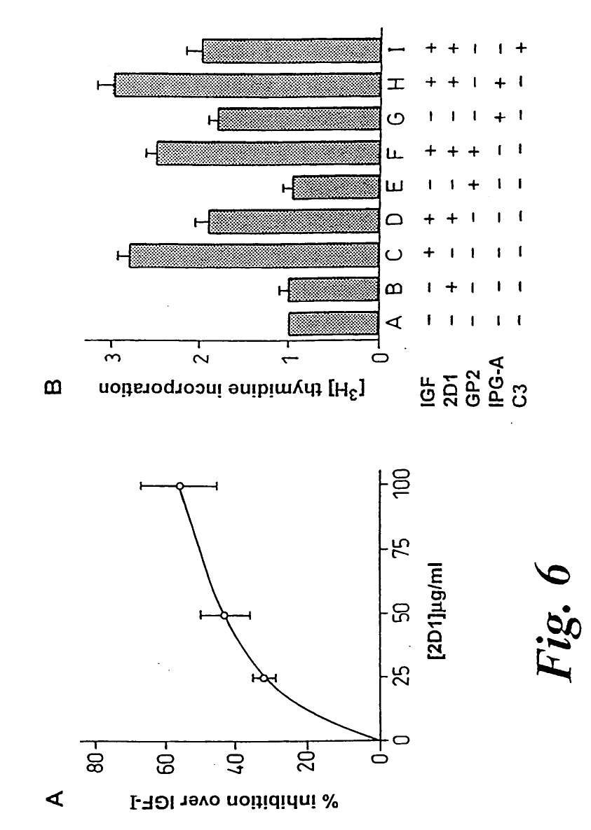

FIG. 6A shows percent of inhibition of 2D1 antibody on CVG cultured in presence of IGF-I. In these experiments, CVGs were isolated as described above. The DNA synthesis was determined by culturing the CVG explants in M199 medium containing 10 μCi/ml of [³H]thymidine of 24 hours. Radioactivity incorporated by the explants was extracted with 10% trichloroacetic acid and quantified by scintillation counting. (A) Percent of inhibition in CVGs cultured with 1 nM IGF-I in presence of increasing concentrations of 2D1 antibody from 25 to 100 μg/ml. (B) Acid-precipitable [³H] thymidine uptake into CVGs cultured in parallel experiments with the additions indicated in FIGS. 5(A-H). The exogenous addition of 100 μM of synthetic analogue C3 was unable to rescue the 2D1 inhibitory effect on CVG growth induced by IGF-I (I). Data are representative of at least 3 different experiment with an average of 4 CVGs per condition.

### EXAMPLE 6

Properties of Anti-IPG Antibody 5H6

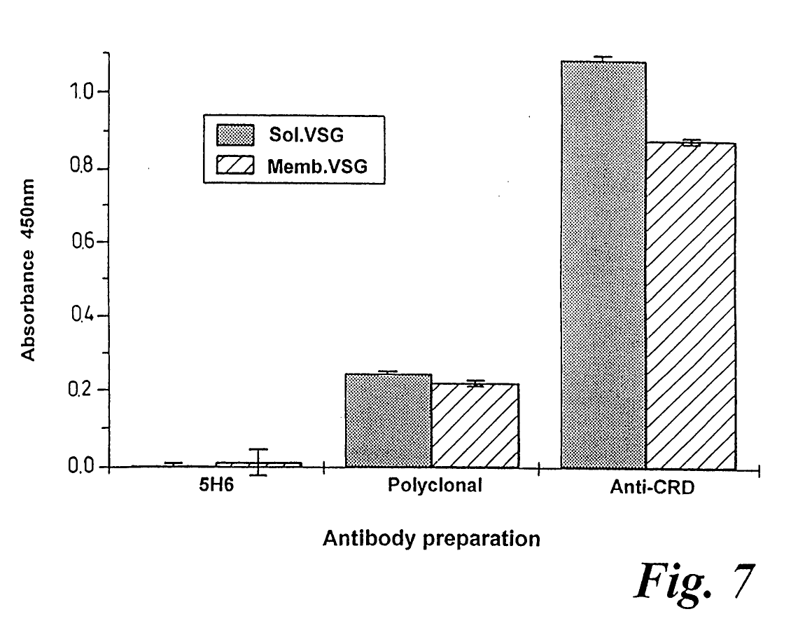

As discussed above, a prior art polyclonal antibody raised against the GPI-anchor of VSG was found to cross-react with IPG containing fractions. FIG. 7 shows that a polyclonal antibody raised against IPGs cross reacted with GPI-anchors. Thus, these results are consistent and demonstrate that there is some cross reactivity between the IPG and GPI anchors. In contrast, when the reactivity of monoclonal antibody 5H6 towards soluble and membrane bound VSG was tested, FIG. 7 (left hand column) shows that it does not cross react in the same way as the polyclonal antibody raised against IPGs (middle column). In these experiments, a polyclonal antibody raised against the common reactive determinant (CRD) common to GPI anchored proteins was used as a positive control (right hand column).

Thus, FIG. 7 shows that antibody 5H6 does not cross react with either soluble or membrane bound VSG, whereas both polyclonal antibodies react strongly. This property distinguishes the antibodies of the invention from cross reacting polyclonal antibodies. This in turn suggests that polyclonal antibodies will not specifically detect IPGs, but are a valuable reagent in ELISA sandwich assays which require two anti-IPG antibodies, one of which is very specific (monoclonal) and the other of general reactivity (polyclonal).

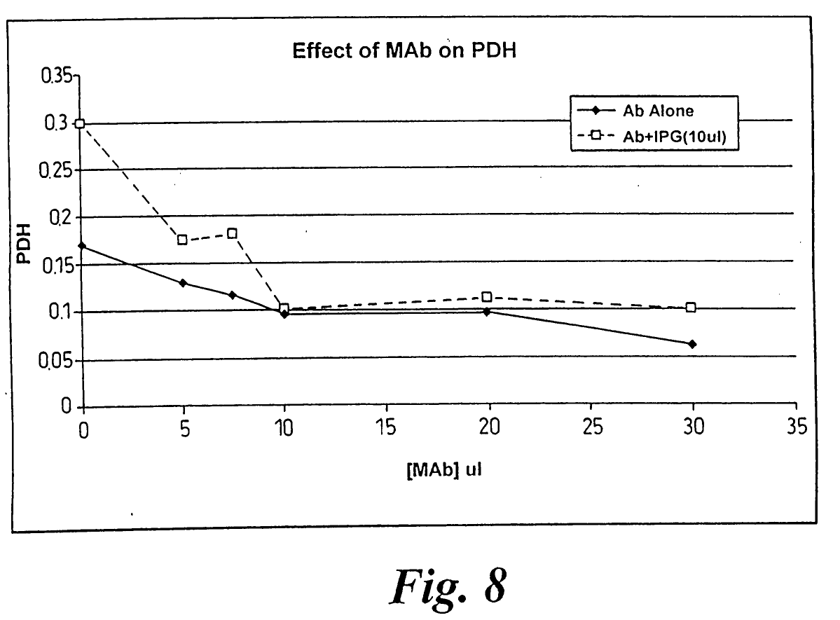

FIG. 8 shows that monoclonal antibody 5H6 is able to inhibit the action of P-type IPG from rat liver in a P-type phosphatase assay. Thus, this and similar antibodies could be used as P-type IPG antagonists, e.g. in the treatment of conditions associated with elevated levels of P-type IPGs such as pre-eclampsia.

### EXAMPLE 8

Assay for Urine IPG Levels (e.g. for use in the diagnosis of pre-eclampsia) I

The following assay was carried out using anti-IPG monoclonal 2D1-the capture antibody and polyclonal rabbit IgG anti-IPG as the secondary antibody. The assay was carried out using samples from pre-eclamptic and control pregnant patient urine samples.

A Maxisorb plate was coated with 50 ml of 2D1 antibody at 2.5 mg/ml in PBS and incubated overnight at 4° C. The plate was then blocked with 200 ml of blocking reagent and incubated for 1 h at 37° C. in a sealed Container floating on a waterbath.

50 ml/well of the urine sample diluted 20 fold in blocking reagent was added and incubated for 2 h at 37° C. The wells were then emptied and washed 5 times with approximately 100 ml of 0.05% Tween20/PBS. 50 ml/well of 2 mg/ml polyclonal rabbit IgG anti-IPG diluted 1/500 in blocking reagent was then added and incubated for 1.5 h 37° C. The wells were then washed 5 times with 100 ml of 0.05% Tween20/PBS and 50 ml/well of goat anti-rabbit IgG-HRP diluted 1/6000 in blocking reagent was added. This was left to incubate for 1 h at 37° C. and wells washed again before adding 50 ml of room temperature pre-warmed TMB solution/well. This was allowed to incubate for 10 minutes at room temperature, after which the colour reaction was topped with the addition of 50 ml of 1 M HCl. The result of the assay was obtained by reading absorbance at 450 nm.

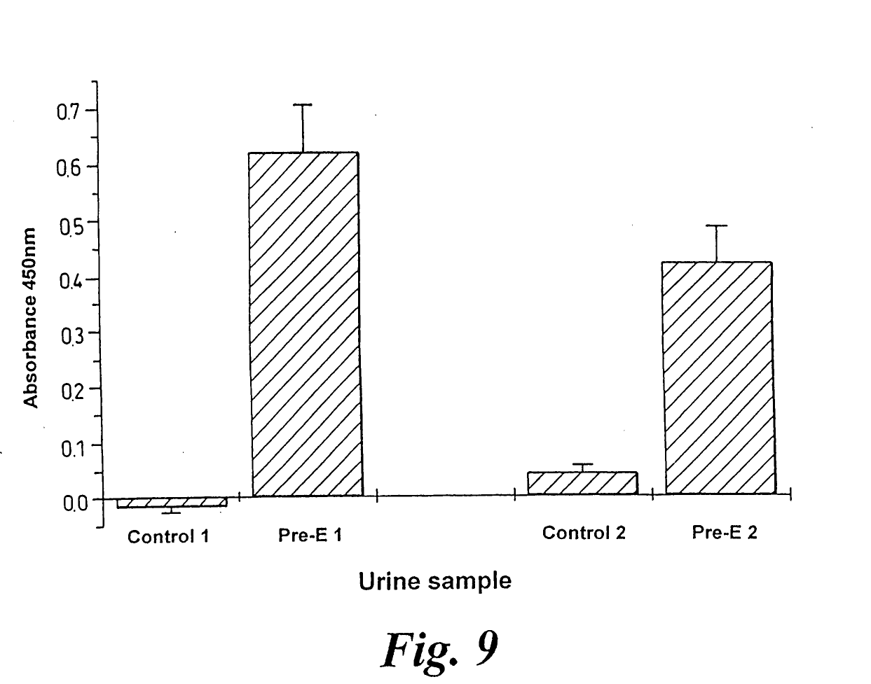

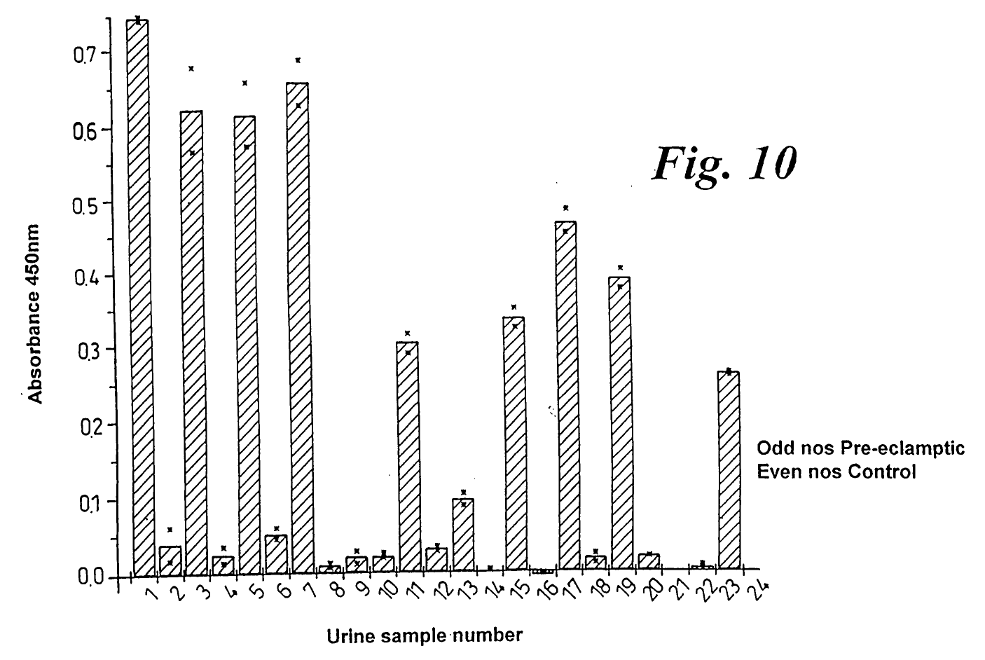

FIG. 9 shows results from the assay comparing two samples from pre-eclamptic patients with non-pre-eclamptic controls. FIG. 10 shows the results obtained from a blinded study of 24 samples, 12 from pre-eclamptic patients. This shows that the assay successfully identified 10/12 of the pre-eclamptic samples with no false positives.

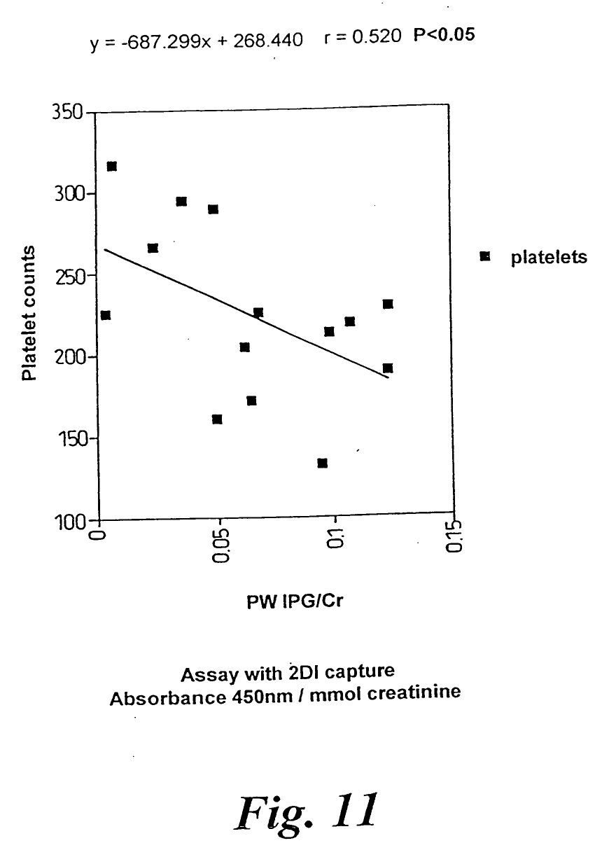

FIG. 11 shows the correlation between platelet counts and the reaction of 2D1 monclonal antibody with urine from pre-eclamptic women. The inverse correlation is significant p<0.05. In pre-eclamptic women, there is a decrease in platelet counts. The figure demonstrates that the higher the level of 2D1 reactive material, the lower the platelet counts, suggesting that the 2D1 antibody is detecting a factor which correlates with pathology in pre-eclamptic women.

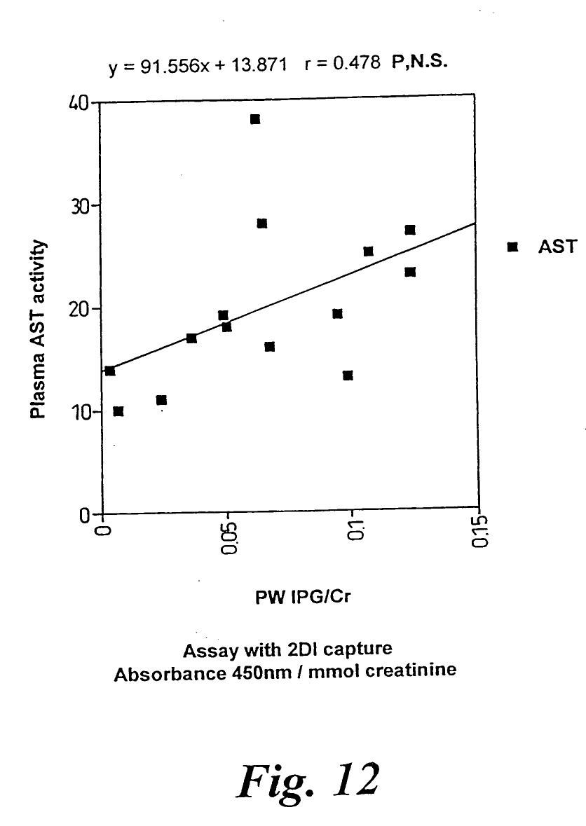

FIG. 12 shows the correlation between plasma aspartic transaminase (AST) and the binding reactivity of 2D1 to urine from pre-eclamptic women. AST (liver derived) levels are elevated in women with pre-eclampsia and the correlation suggests that the IPG levels may correlate with pathology in the disease.

Deposits

The deposit of hybridomas 2F7, 2D1 and 5H6 in support of this application was made at the European Collection of Cell Cultures (ECACC) under the Budapest Treaty by Rademacher Group Limited (RGL), The Windeyer Building, 46 Cleveland Street, London W1P 6DB, UK. The deposits have been accorded accession numbers accession numbers 98051201, 98031212 and 98030901. RGL give their unreserved and irrevocable consent to the materials being made available to the public in accordance with appropriate national laws governing the deposit of these materials, such as Rules 28 and 28a EPC. The expert solution under Rule 28(4) EPC is also hereby requested.

References:

The reference mentioned herein are all incorporated by reference in their entirety.

Caro et al, Biochem. Molec. Med., 61:214-228, 1997.

Kunjara et al, In: Biopolymers and Bioproducts: Structure, Function and Applications, Ed Svati et al, 301-305, 1995.

Rademacher et al, Brazilian J. Med. Biol. Res., 27:327-341, 1994.

Nestler et al, Endocrinology, 129:2951-2956, 1991.

Romero et al, P.N.A.S., 87:1476-1480, 1990.

Huang et al, Endocrinology, 132:652-657, 1993.

Represa et al, P.N.A.S., 88:8016-8019, 1991.

Varela-Nieto et al, Dev. Biol., 143:432-435, 1991.

Mato et al, J. Biol. Chem., 262:2131-2137, 1987.

Clemente et al, Cell Signalling, 7:411-421, 1991.

Varela-Nieto et al, In Handbook of Endocrine Research Techniques: Intracellular Mediators of Peptide Hormone Action: Glycosylphospatidylinositol/Inositol Phosphoglycan System, ed de Pablo et al, San Diego: Academic Press, 391-406, 1993.

Avila et al, Biochem. J., 282:681-686, 1992.

Gaulton, Diabetes, 40:1297-1304, 1991.

WO98/011116 and WO98/011117 (Rademacher Group Limited)

1-25. (Cancelled)

26. A hybridoma cell line which produces an anti-inositol phosphoglycan (IPG) monoclonal antibody, wherein the cell line is selected from the group of hybridoma cell lines consisting of 2F7, 2D1 and 5H6 deposited at European Collection of Cell Cultures (ECACC) under accession numbers 98051201, 98031212 and 98030901.

27. An anti-IPG monoclonal antibody from a hybridoma cell line of claim 26.

28. An anti-IPG monoclonal antibody which is capable of binding to an epitope of an IPG, which epitope is bound by a monoclonal antibody of claim 27.

29. The anti-IPG antibody of claim 28 wherein the antibody is capable of binding an epitope present in A-type IPG having a structure identical to an A-type IPG obtained from rat liver and P-type IPG having a structure identical to a P-type IPG obtained from human placenta.

30. The anti-IPG monoclonal antibody of claim 27, wherein the antibody does not substantially bind a common reactive determinant (CRD) of GPI anchored proteins.

31. The anti-IPG antibody of claim 27, wherein the antibody neutralises an IPG biological activity.

32. A composition comprising the anti-IPG monoclonal antibody of claim 27.

33. A method of producing an anti-IPG antibody comprising culturing a hybridoma cell line of claim 26 and isolating the antibody thus produced.

34. A method of determining whether a patient is at risk of developing type I diabetes, the method comprising:
(a) contacting a biological sample from the patient with an antibody capable of specifically binding inositol phosphoglycans (IPGs) present in the sample; and,
(b) determining the binding of IPGs present in the sample to the anti-IPG antibody;
(c) comparing the binding of the IPGs determined in step (b) to control binding to determine whether the patient is at risk of developing type I diabetes.

35. The method of claim 34 which further comprises an initial step of providing the patient with a carbohydrate load and determining the IPGs present in the sample following the carbohydrate load over time to obtain an IPG profile.

36. The method of claim 35, wherein the IPG profile determined from the patient sample is compared with profiles obtained from normal subjects and patients having type I diabetes to determine whether the patient is at risk of developing type I diabetes.

37. The method of claim 34 which further comprises administering insulin and/or IPGs to a patient determined to have or be at risk of developing type I diabetes.

38. The method of claim 34, wherein the anti-IPG antibody is immobilised on a solid support.

39. A method of purifying inositol phosphoglycans from a sample comprising IPGs, the method comprising:
(a) contacting the monoclonal antibody of any one of claims 27 to 29 with the sample comprising IPGs so that the monoclonal antibody binds to the IPGs present in the sample; and,
(b) separating the monoclonal antibody and the bound IPG from other sample components.

40. A method of producing anti-IPG antibodies, the method comprising immunising an animal with one or more soluble IPGs or a lipid conjugate thereof to elicit an antibody response, wherein the IPGs are not conjugated to an immunogenic carrier.

41. The method of claim 40 wherein the animal is immunised via an intraperitoneal route using said one or more soluble IPGs.

42. A method of determining whether a patient has or is at risk of developing pre-eclampsia, the method comprising:
(a) contacting a biological sample from the patient with an antibody of any one claims 27 to29, wherein the antibody is capable of specifically binding P-type inositol phosphoglycans (IPGs) present in the sample; and,
(b) determining the binding of IPGs present in the sample to the anti-IPG antibody; and,
(c) comparing the binding of the IPGs determined in step (b) to control binding to determine whether the patient is at risk of developing pre-eclampsia.

43. A method of producing a labelled reagent capable of binding to inositol phosphoglycans (IPGs), the method comprising conjugating the antibody of any one of claims 27 to 29 to a label or reporter molecule, wherein the label or reporter molecule is capable of directly or indirectly generating a detectable signal.

44. The method of claim 43, wherein label is selected from the group consisting of a fluorescent label, a radioactive label or an enzyme label.

45. A solid support comprising an antibody of any one of claims 27 to 29 immobilized thereon.

46. A composition comprising the anti-IPG monoclonal antibody of claim 28.

47. A composition comprising the anti-IPG monoclonal antibody of claim 29.

48. A labelled reagent capable of binding to inositol phosphoglycans (IPGs) comprising the antibody of any one of claims 27 to 29 conjugated to a label or reporter molecule, wherein the label or reporter molecule is capable of directly or indirectly generating a detectable signal.
